(19) United States
(12) Patent Application Publication
(10) Pub. No.: US 2012/0158133 A1
(43) Pub. Date: Jun. 21, 2012

(54) Intraocular Lens

(75) Inventor: Wolfgang Mueller-Lierheim, Munich (DE)

(73) Assignee: Miro GmbH, Munchen (DE)

(21) Appl. No.: 13/392,853

(22) PCT Filed: Aug. 25, 2010
(86) PCT No.: PCT/EP2010/005212

§ 371 Date: Feb. 27, 2012

(30) Foreign Application Priority Data
Aug. 28, 2009 (DE) 202009011716.2

(51) Int. Cl. A61F 2/16

(52) U.S. Cl. 623/6.56

(57) ABSTRACT

An intraocular lens comprising a hydrophobic lens body (1), at the surface of which there is provided a hydrophilic layer (2) to achieve a reflection-free transition of the incident light.

The invention concerns an intraocular lens. Lenses of that kind are implanted in particular after clouding of the natural lens of the eye as a replacement for the natural lens in the context of a cataract operation. Conventionally the lens body comprises hydrophobic material, in particular copolymers, containing acrylate and/or methacrylate. To reduce stickiness it is also known to add fluorinated acrylate or methacrylate to the lens material (WO 2007/062864). It is also known to use for the intraocular lens, a lens material which has a high refractive index to permit a small lens thickness to be achieved. Lenses of that kind which in particular are folded or rollable can then be implanted in the eye through relatively small incisions by means of injectors as are known for example from U.S. Pat. No. 6,355,046 B2.

Because of the difference in refractive index of the lens material in relation to the surrounding medium in the eye, in particular the aqueous humour in the anterior chamber of the eye and vitreous humour at the rear of the lens light reflection occurs at the interfaces. That is correspondingly more pronounced, the greater the difference in refractive index between the lens material and the surrounding medium.

In addition, in the production of the intraocular lenses the surfaces are cuttingly machined by means of submicrometer technology to avoid a subsequent polishing step. The lens surfaces treated in that way however still suffer from micro-roughnesses which can contribute to light scatter.

The problem of the present invention is to provide an intraocular lens in which a light-optical transition which is as continuous as possible takes place between the lens surface and the medium surrounding the lens implanted in the eye.

That problem is solved by the intraocular lens having the features of claim 1.

Such a lens has a hydrophobic lens body, at the surface of which there is provided a hydrophilic layer. Preferably the lens body comprises a hydrophobic, foldable polymer material comprising at least one acrylate and/or methacrylate, a fluorinated acrylate or methacrylate can be added to reduce stickiness. Suitable lens materials are known for example from WO 2007/062864 A2. In addition silicone rubber can be used as the hydrophobic material for the lens body.

The hydrophilic layer comprises at least one hydrophilic acrylate with good tissue and blood compatibility. That coating prevents the adhesion of fibrin and adherent cells and thereby opposes post-operative membrane formation (secondary cataract, PCO=posterior capsular opacification).

The hydrophobic lens material is a material which absorbs less than 5% by volume of water. Preferably this involves a foldable or rollable lens body. It is however also possible to use lens bodies comprising a rigid material, for example polymethyl methacrylate (PMMA), for the lens body.

The lens bodies can involve those whose surface is produced by a cutting procedure by means of submicrometer technology and thus has micro-roughnesses which can contribute to light scatter. Instead of cutting machining the lens bodies and in particular the lens bodies comprising silicone rubber can be produced by the moulding process. In that case the roughness of the mould surfaces is transferred to the lens surface. The roughnesses are smoothed and light scatter very substantially avoided by application of the hydrophilic coating to that surface.

The refractive index of the hydrophilic coating is so selected that it is between the refractive index of the lens material and that of the surrounding medium in the eye, which are substantially aqueous humour and vitreous humour. That means that the refractive index can preferably be selected to be between n=1.336 (aqueous humour) and 1.38 (vitreous humour) respectively and n=1.56 (polymer material known from WO 2007/062864 A2). As the hydrophilic coating, in particular hydrophilic acrylate layer, is between the hydrophobic lens material constituting the lens body and the surrounding medium in the eye, namely that of the aqueous humour and the vitreous humour, that affords an approximately continuous light-optical transition between the aqueous humour, the lens body and the vitreous humour, whereby light reflection and light scatter caused by the remaining micro-roughnesses can be reduced or avoided.

In addition the hydrophilic coating improves the sliding property of the lens upon implantation by means of an injector or another implantation tool, for example tweezers. Such injectors are known for example from U.S. Pat. No. 6,355,046 B2 and serve for holding or rolling the lens to be implanted. In the implantation operation the folded or rolled lens is implanted into the eye through a cannula which is fitted through a minimal opening in the eye.

As the inside wall of the cannula and the folding cartridge which are provided in the known injectors generally comprise polyethylene or polypropylene the sliding property against the inside wall of the cartridge and the cannula is relatively slight so that the injection operation is made more difficult without additional lubricants. The lubricants used can get into the eye in that case.

According to the invention those disadvantages can be eliminated by a hydrophilic acrylate layer applied to the inside wall of the folding cartridge and the cannula.

Accordingly the invention also proposes an implantation set which comprises the above-described intraocular lens and an injector for implanting the intraocular lens. The intraocular lens can possibly be stored in the injector, in which case the injector represents a disposable component. By virtue of the hydrophilic coating at the intraocular lens and/or the inside wall of the injection cannula and possibly the folding cartridge of the injector, that affords an improvement in the sliding properties and thus facilitates implantation of the intraocular lens.

The invention is described in greater detail by means of embodiments by way of example with reference to the Figures in which:

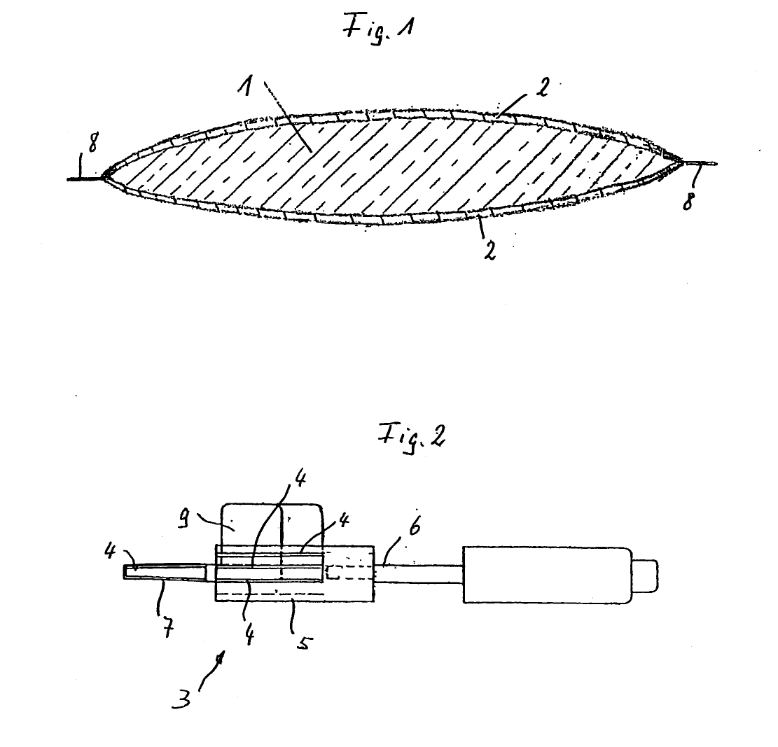

FIG. 1 diagrammatically shows a sectional view of an intraocular lens as an embodiment of the invention, and

FIG. 2 shows an embodiment for an injector with which an intraocular lens can be implanted in an eye.

The embodiment shown in FIG. 1 of an intraocular lens has a lens body 1 of hydrophobic material. The hydrophobic material can be formed from a copolymer material having at least one acrylate and/or methacrylate. Preferably the lens material is such that the lens body 1 can be folded or rolled. It is however also possible for the lens body to be made from a rigid material, for example polymethyl methacrylate (PMMA). The surface of the lens body 1 has a micro-roughness which can be caused by the cutting manufacturing process in particular using submicrometer technology. A hydrophilic layer 2 of a hydrophilic acrylate is applied to the micro-rough surface of the lens body 1. That hydrophilic acrylate can be for example dihydroxypropyl methacrylate (DHPMA). The coating operation is effected by means of a coating process to be described hereinafter. A haptic 8 in the form of threads or support limbs or in the form of a support border entirely or partially extending around the lens body 1 can be provided at the edge of the optical lens body.

An intraocular lens injector of conventional structure as is known for example from U.S. Pat. No. 6,355,046 B2 is suitable for that implantation of such an intraocular lens.

The embodiment of an injector shown in FIG. 2 is of substantially a conventional structure. It comprises a folding cartridge 5 in which the intraocular lens can firstly be stored in the non-folded state. For the implantation procedure the lens in the lens cartridge 5 is folded or rolled for example by means of wings 9 which can be folded together so that its diameter is reduced to the inside diameter of a cannula 7. The folded or rolled lens is pushed out of the cartridge 5 by means of a pushrod 6 through the cannula 7 which is passed through a small incision on the eye, and is implanted in the eye. In that case the intraocular lens is fitted into the previously opened lens capsule. To improve the sliding properties the inside walls of the folding cartridge 5, that come into contact with the surface of the lens, and the inside wall of the cannula 7, are fitted with a hydrophilic coating 4 which is identical to the hydrophilic coating 2 on the intraocular lens.

An embodiment of the intraocular lens according to the invention is described in greater detail hereinafter.

The hydrophilic layer 2 is applied to the lens body 1 of a hydrophobic material, for example PMMA or a hydrophobic lens material known from WO 2007/062864 A2, after activation of the surface of the lens body 1. Activation of the surface of the lens body 1 can be effected by saponification, for example with sodium hydroxide, whereby the surface of the lens body is negatively charged. Activation of the surface can also be effected by plasma activation, for example using a nitrogen plasma. The monomer of the hydrophilic acrylate, for example dihydroxypropyl methacrylate (DHPMA) is applied to the activated surface with a light initiator, in particular a UV initiator, for example by means of ethyl acetate in solution. Tetrahydrofuran is suitable as a further solvent. After evaporation of the solvent polymerisation is effected by means of radiation, in particular UV radiation. The surface is then washed and vacuum-dried at about 35° C.

That results in an interpenetrating polymer network in a surface region of the hydrophobic lens material. It occurs in particular in respect of surface swelling of the hydrophobic lens material due to the solvent with which the hydrophilic acrylate is applied in the form of a layer to the surface of the lens body. In that case molecules of the hydrophilic acrylate are incorporated into the surface of the hydrophobic lens material and cross-linked after polymerisation therewith. The above-described activation of the surface of the lens body further promotes that process. As a result an intermediate layer with a refractive index which is between that of the hydrophilic layer and that of the lens body is produced at the surface of the lens body with the interpenetrating network between the core of the lens body and the hydrophilic layer on the surface of the lens body. In that way, in contrast to the coating known from U.S. 2009/0018651 A1 and comprising one or more layers between the lens body material and the coating without an additional coating, that affords a reflection-free transition between the coating and the lens body material.

The formation of that interpenetrating polymer network is promoted by the described activation of the surface and also by the radiation, in particular UV radiation. That affords a permanent polymer network.

A further improvement in the continuous transition of the refractive index between the aqueous humour of the eye and the hydrophobic lens body of the intraocular lens can also be achieved by the material of the hydrophilic layer being swollen by the aqueous humour. In the swollen surface region of the hydrophilic layer there is a refractive index between that of the aqueous humour and that of the hydrophilic layer. That therefore achieves a reflection-free transition between the aqueous humour and the hydrophilic layer on the lens body.

In the same manner the surfaces of the injector shown in FIG. 2 can also be provided with the hydrophilic coating.

### LIST OF REFERENCES

1. Hydrophobic lens body
2. Hydrophilic layer
3. IOL injector
4. Hydrophilic coating
5. Folding cartridge
6. Pushrod
7. Cannula
8. Lens haptic
9. Folding wings

1. An intraocular lens comprising a hydrophobic lens body characterised in that a hydrophilic layer is provided at the surface of the lens body.

2. An intraocular lens according to claim 1 wherein the lens body comprises a polymer material which is formed from at least one hydrophobic acrylate and/or hydrophobic methacrylate or silicone rubber.

3. An intraocular lens according to claim 1 wherein the hydrophilic layer is formed from hydrophilic acrylate.

4. An intraocular lens according to claim 1 wherein a refractive index of the hydrophilic layer is between that of the hydrophobic lens body and that of an aqueous humour of an anterior chamber of at least one of an eye and a vitreous humour of the eye.

5. An intraocular lens according to claim 1 wherein at its surface the lens body has a micro-roughness resulting from cutting lens production or moulding of the lens.

6. An intraocular lens according to claim 1 wherein the lens body is foldable or rollable.

7. An intraocular lens according to claim 1 wherein molecules of the hydrophilic layer are incorporated into a surface layer of the lens body.

8. An intraocular lens according to claim 7 wherein the incorporated molecules of the hydrophilic layer are cross-linked with molecules of the lens body.

9. An intraocular lens according to claim 7 wherein the surface layer of the lens body in which the molecules of the hydrophilic layer are incorporated has a refractive index between that of the hydrophilic layer and the lens body.

10. An intraocular lens according to claim 1 wherein the hydrophilic layer is swellable by an aqueous humour and a refractive index of a swollen surface region of the hydrophilic layer is between that of the aqueous humour and the hydrophilic layer.

11. An intraocular lens according to claim 3 wherein the hydrophilic acrylate is dihydropropyl methacrylate.

12. An intraocular lens according to claim 1 wherein the intraocular lens is configured to be used as a component part of an injector set having an injector with which the lens is to be implanted, wherein the injector at least at its inside wall along which the lens is guided in the implantation procedure has a hydrophilic acrylate coating.
